(19) United States
(12) Patent Application Publication
(10) Pub. No.: US 2021/0244495 A1
(43) Pub. Date: Aug. 12, 2021

(54) Marker Systems and Methods

(71) Applicant: SHADWELL ENDOSCOPIC, INC., Weston, MA (US)

(72) Inventors: Adam Scott Landsman, Weston, MA (US); Paul Vonryll Gryska, Chatham, MA (US); Joseph Ferrucci, Weston, MA (US); William Densel, Sudbury, MA (US); Robert F. Rioux, Ashland, MA (US)

(21) Appl. No.: 17/168,732

(22) Filed: Feb. 5, 2021

Related U.S. Application Data
(60) Provisional application No. 62/971,652, filed on Feb. 7, 2020.

(51) Int. Cl. A61B 90/00; A61B 90/98; A61B 18/08

(52) U.S. Cl. CPC A61B 90/39; A61B 90/98; A61B 17/3468; A61B 2090/3987; A61B 18/082

(57) ABSTRACT

Marker systems and methods for implanting a marker are provided. In one embodiment, a marker system can include an elongate cannulated tube, a marker capsule, and a driver member. The cannulated tube can have a piercing feature arranged at a distal end thereof that is configured to create a working portal through tissue such that fluid can be delivered into the tissue to form a bleb. The marker capsule can be removably disposed within the lumen of the cannulated tube. The marker capsule can include a transmission antenna, and an arranging feature disposed on an external surface configured to facilitate rotation of the marker capsule within the bleb. The driver member can be arranged within the lumen of the cannulated tube proximal to the marker capsule. The drive member can be configured to advance distally to displace the marker capsule through the working portal and into the bleb.

### CROSS-REFERENCE TO RELATED APPLICATIONS

The present application claim priority to U.S. Application No. 62/971,652, filed on Feb. 7, 2020 and entitled “SYSTEMS AND METHODS FOR MARKING AND LOCATING SITES OF BIOPSIES USING RADIO FREQUENCY IDENTIFICATION TRANSPONDER,” which is hereby incorporated by reference in its entirety.

### FIELD

The present application relates to surgical marker devices and methods for implanting surgical marker devices.

### BACKGROUND

During examinations of the intestine, an endoscope is used to visualize the lining of the intestines of a subject. Typically when a polyp or abnormal tissue is encountered, under visualization with the endoscope, the polyp is grasped by a tool arranged on the endoscope and removed from the wall of the intestine. It is generally important at the time of removal of a polyp to mark the location of the removed tissue on the lining of the intestines. A resection or other medical procedures may be required to be performed at the marked location of the polyp removal, depending on the results of a biopsy of the polyp or abnormal tissue, medical recommendations, or pursuant to other follow-up procedures.

Historically, these marks to the intestinal wall have been achieved using a liquid stain/dye injected onto the intestinal wall to create a contrasting mark. This has been viewed as a relatively imprecise method due to the fact that the dye may be released over large areas of the intestinal wall, which may cause excessive streaking, rather than placing the dye within the wall of the intestine. Furthermore, it is possible for the dye to flow to adjacent areas of the polyp removal site, making the marked location of the polyp removal site less precise. Due to the imprecision of the dying process, it is frequently difficult to identify and differentiate one marked site from the next if multiple polyp removal sites are in close proximity. Also, the dye used in the marking procedure is not radiopaque, and therefore, may make it extremely difficult to locate the marked polyp removal site when returning to the subject for further resection, either by colonoscopy or by laparoscopic surgery. Even further, dye used in marking procedures can fade over time, preventing identification of the previously marked site.

An alternative technique to using a dye involves securing a metallic clip marker to the mucosal layer (i.e., the inner wall of the intestine). In many cases, it is desirable for a user to locate the site of a previous soft tissue/polyp removal, especially if the need to locate the previous removal site occurs frequently. However a potential complication of this method is that the metallic clip marker can break loose from the mucosal layer, which leaves the removal site unmarked. In particular, since metallic clip markers are clamped to the surface of the inner wall of the intestine, the clip markers may become dislodged due to normal bodily activity, such as when waste products pass through the intestine. As a result, clip markers may become dislodged from the inner intestinal wall with little to no warning to the subject. Dislodgement of the clip marker may interfere or impede subsequent location and re-examination of the site of a soft tissue removal. In many cases, it is critical for a user to locate and re-examine the site of a soft tissue removal year after year. Other techniques for tracking the location of a soft tissue removal site may include measuring the distance from a fixed point on the subject, such as the anus, to the soft tissue removal site along the intestines. However, this technique has also proved to be highly inaccurate, especially after a prior intestinal resection.

A soft tissue removal site that was marked during a previous colonoscopy (i.e., internal to the intestine) may have to be relocated laparoscopically (i.e., external to the intestine within the peritoneal cavity). Therefore the marker that is used to mark the site during the previous colonoscopy must be able to be found when working from the exterior of the intestinal wall. This may be difficult to do when a portion of the intestine has moved or folded on itself, since there are no visual means of identification visible, such as during a laparoscopic procedure.

### SUMMARY

Implementations of the present disclosure provide marker systems and corresponding methods for implanting a marker in tissue.

In one embodiment, a marker system is provided having an elongate cannulated tube, a marker capsule, and a driver member. The cannulated tube has a lumen extending therethrough and can have a piercing feature arranged at a distal end of the tube that is configured to create a working portal through tissue such that fluid can be delivered into the tissue to form a bleb. The marker capsule can be removably disposed within the lumen of the cannulated tube, and it can include a transmission antenna, and an arranging feature disposed on an external surface of the capsule and configured to facilitate rotation of the marker capsule within the bleb. The driver member can be arranged within the lumen of the cannulated tube proximal to the marker capsule, and it can be configured to advance distally to displace the marker capsule through the working portal and into the bleb.

The arranging feature can have a variety of configurations, and the marker can include any number of arranging features. In one embodiment, the arranging feature can be in the form of a fin extending from the external surface of the marker capsule. The driver member can include jaws configured to grasp the fin to rotate the marker capsule. In another embodiment, the arranging feature can be in the form of a notch arranged on a distal end of the marker capsule and a thread arranged within the notch and configured to apply a tension to the marker capsule to rotate the marker capsule. In another embodiment, the arranging feature can be in the form of a threaded loop arranged on a distal end of the marker capsule and configured to apply tension to the marker capsule to rotate the marker capsule. The marker can include any combination of the aforementioned arranging features.

In another embodiment, a marker system is provided and includes an elongate cannulated tube, a marker capsule, a driver member, and a cannulated needle. The elongate cannulated tube can have a lumen extending through the tube, with a distal end of the tube including a hook-shaped member with an internal curved surface. The marker capsule can be removably disposed within the lumen of the cannulated tube, and it can include a transmission antenna. The driver member can be arranged within the lumen of the cannulated tube proximal to the marker capsule, and it can be configured to be advanced distally to advance the marker capsule along the internal curved surface of the hook-shaped member such that the marker capsule is caused to rotate. The cannulated needle can be arranged along the elongate cannulated tube and it can be configured to be slidably advanced from a distal end of the elongate cannulate tube to penetrate tissue.

The marker system can have a variety of configurations and can include various features. In one implementation, the cannulated needle can be slidably disposed through a second lumen formed in a sidewall of the cannulated tube. In other aspects, the hook-shaped member can be positioned to block distal advancement of the marker capsule, and the hook-shaped member can be flexible to deflect when the marker capsule is advanced distally. In another implementation, the marker system can include a tether coupled to the hook-shaped member and configured to apply a force to the hook-shaped member to cause the hook-shaped member to deflect and thereby allow distal advancement of the marker capsule.

In another embodiment, a marker system is provided having an elongate cannulated tube, a marker capsule, a thread, and a driver member. The elongate cannulated tube can have a lumen extending through the tube, and the marker capsule can be removably disposed within the lumen of the cannulated tube. The marker capsule can have a notch formed in a distal surface of the capsule, and a transmission antenna can be disposed within the capsule. The thread can be arranged within the notch of the marker capsule. The driver member can be arranged within the lumen of the cannulated tube proximal to the marker capsule, and it can be configured to distally advance to displace the marker capsule from the distal end of the cannulated tube.

The thread can have a variety of configurations. In one implementation, the thread can be coupled to an energy source and it can be configured to apply an electrical energy to a tissue surface to create a working portal. The thread can also be configured to apply tension to the marker capsule to rotate the marker capsule.

In other aspects, the marker system can include a receiver member have a short-range transmission receiver and a long-range transmission antenna. The receiver member can be configured to receive a data set from the marker capsule via the short-range transmission receiver, and it can be configured to transmit the data set via the long-range transmission antenna. The receiver member can be disposed within a water-proof cap configured to couple to an end of an endoscope. In one embodiment, the short-range transmission receiver can be a RFID receiver, and the long-range transmission can be a short-link radio antenna.

Methods for implanting a marker in tissue are also provided. In one embodiment, the method can include inserting an elongate cannulated tube into a tissue surface to create a working portal through the tissue surface. A fluid can be injected through the cannulated tube into the tissue surface to create a bleb. A marker capsule can be ejected from a distal end of the cannulated needle into the bleb. The marker capsule can have a transmission antenna within the marker capsule. The marker capsule can be rotated within the bleb such that a length of the marker capsule extends adjacent to the working portal to prevent passage of the marker capsule through the working portal.

In one embodiment, the fluid can be injecting through a cannulated needle extending through the cannulated tube. The method can also include, prior to inserting the elongate cannulated tube into a tissue surface, advancing a needle from a distal end of the cannulated tube. The cannulated needle can be removed from the bleb prior to rotation of the marker capsule.

In another embodiment, rotating the marker capsule can include applying tension to a tether coupled to the marker capsule to cause the marker capsule to rotate. In other aspects, rotating the marker capsule can include grasping a fine on the marker capsule with a grasper and manipulating the grasper to rotate the marker capsule. The method can also include positioning a receiver member adjacent to the marker capsule such that the receiver member receives a data set from the marker capsule via the short-range transmission receiver.

### BRIEF DESCRIPTION OF THE DRAWINGS

These and other features will be more readily understood from the following detailed description taken in conjunction with the accompanying drawings, in which:

FIG. 1 is a perspective view of one exemplary embodiment of a marker system including a marker capsule;

FIG. 2A is a side view of the marker capsule of FIG. 1, showing a notch and a fin on the marker capsule;

FIG. 2B is a side view of another embodiment of a marker capsule for use with the marker system of FIG. 1;

FIG. 2C is a side view of another embodiment of a marker capsule for use with the marker system of FIG. 1;

FIG. 2D is a side view of yet another embodiment of a marker capsule for use with the marker system of FIG. 1;

FIG. 3A is a schematic view showing the marker system of FIG. 1 extending through a body lumen;

FIG. 3B is a schematic view showing a needle of the marker system of FIG. 3A advanced through tissue for injecting saline to form a bleb;

FIG. 3C is a schematic view showing a marker being ejected from the marker system of FIG. 3B into the bleb;

FIG. 3D is a schematic view showing a driver member of the marker system of FIG. 3C grasping a fin on the marker and rotating the marker;

FIG. 3E is a schematic view showing the driver member of the marker system of FIG. 3D applying tension to a thread coupled to the marker for rotating the marker;

FIG. 3F is a schematic view showing the marker system of FIG. 3E removed from the bleb and closing an opening of the bleb;

FIG. 4 is a schematic view showing an electrical thread on a distal tip of a marker system being abutting into tissue;

FIG. 5A is a schematic view showing a needle on another embodiment of a marker system being advanced into tissue for delivering fluid to form a bleb;

FIG. 5B is a schematic view showing a flexible tip of the marker system of FIG. 5A deflecting as a marker capsule is advanced from the tip;

FIG. 6A is a perspective view of one exemplary embodiment of an endoscope cap having a receiver member;

FIG. 6B is an cross-sectional view of the endoscope cap of FIG. 6A; and

FIG. 6C is a partial cross-sectional view of the endoscope cap of FIG. 6A arranged on an endoscope.

It is noted that the drawings are not necessarily to scale. The drawings are intended to depict only typical aspects of the subject matter disclosed herein, and therefore should not be considered as limiting the scope of the disclosure.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Various devices and methods are provided for inserting a marker capsule into tissue, and for rotating the marker capsule to prevent the capsule from backing out the working portal made to insert the capsule. The devices and methods disclosed herein are particularly advantageous as they allow a marker capsule to be placed under the surface of the tissue, while also preventing the capsule from being expelled from the tissue after insertion. In certain exemplary embodiments, the marker capsule has features arranged on its exterior surface that facilitate rotation of the capsule once inserted under the tissue. In this way, the marker capsule can be inserted in a first position, having a width smaller than the working portal to aid in insertion, and rotated to a second position, where the length of the capsule is larger than the width of the working portal, preventing backing out of the capsule. With the marker capsule inserted and rotated, the marker capsule can wirelessly transmit data about the insertion site to a receiver member. The data set can include information about the insertion site, such as date, time, etc., and can be located wirelessly during a subsequent procedure, and without the need of visual reference points. This may avoid the deficiencies of the prior art techniques discussed above. By placing the marker capsule beneath the tissue surface, and rotating the capsule to prevent backing out, loss of the marker capsule may be reduced, and identification of the marker capsule during subsequent procedures may also be simplified, thereby shortening the procedure.

Various embodiments of marker systems are disclosed herein. In general, FIG. 1 illustrate one embodiment of a marker system 100 having an elongate cannulated tube 103, which includes an outer shaft 106 and a needle 104 extending therethrough with a piercing feature 105 arranged on a distal end thereof. A marker capsule 108 is removably arranged within the cannulated tube 103. Additionally, a driver member 112 is arranged within the cannulated tube 103, proximal to the marker capsule 108.

In this embodiment, the cannulated tube 103 is disposed through an insertion device 102, which in is in the form of an endoscope having various working channels 103A extending therethrough and a camera 103B on a distal end thereof. The endoscope can aid in facilitating delivering of the marking system into a body lumen. However, a person skilled in the art will appreciate that any delivering device can be used or that the marker system can be advanced into a body lumen without a delivery device.

The elongate cannulated tube 103 can have a variety of configurations, but is generally in the form a flexible member with at least one lumen extending therethrough. As indicated above, the cannulated tube 103 shown in FIG. 1 includes an outer shaft 106 and a needle 104 extending therethrough with a piercing feature 105 arranged on a distal end thereof. The piercing feature can be in the form of a sharp edge configured to penetrate through tissue. The needle 104 can be slidably disposed within the shaft 106 to allow the needle 104 to be retracted into the shaft 106 during delivery, and to allow the needle 104 to be advanced distally beyond the distal end of the shaft 106 for penetration through tissue. Various techniques can be provided to control advancement and retraction of the needle, such as an actuation mechanism on a handle assembly (not shown) at a proximal end of the marker system. In other aspects, the needle 104 can be separable from the shaft 106 104, and can be inserted through the tube once the tube is positioned within a body lumen. In other aspects, the needle 104 can be integral with the shaft 106 such that the needle and shaft and in the form of a single elongate tube having a tissue-penetrating tip.

The driver member 112 extends through the cannulated tube 103, and can have a variety of configurations but is generally in the form a flexible member having an end effector. The driver member 112 can include a flexible section adjacent the distal end for allowing the end effector to be articulated. In order to articulate the end effector, control devices such as steering wires could be run within the driver member 112 to the end effector in order to steer the end effector. The end effector can be a grasper which can actuate its grasping mechanism in order to grab onto the capsule and adjust the capsule, or the end effector can be a pushing mechanism which can apply a pressure to the capsule in order to move the capsule. However, a person skilled in the art will appreciate that a variety of grasper devices are known in the art and can be used to manipulate the capsule.

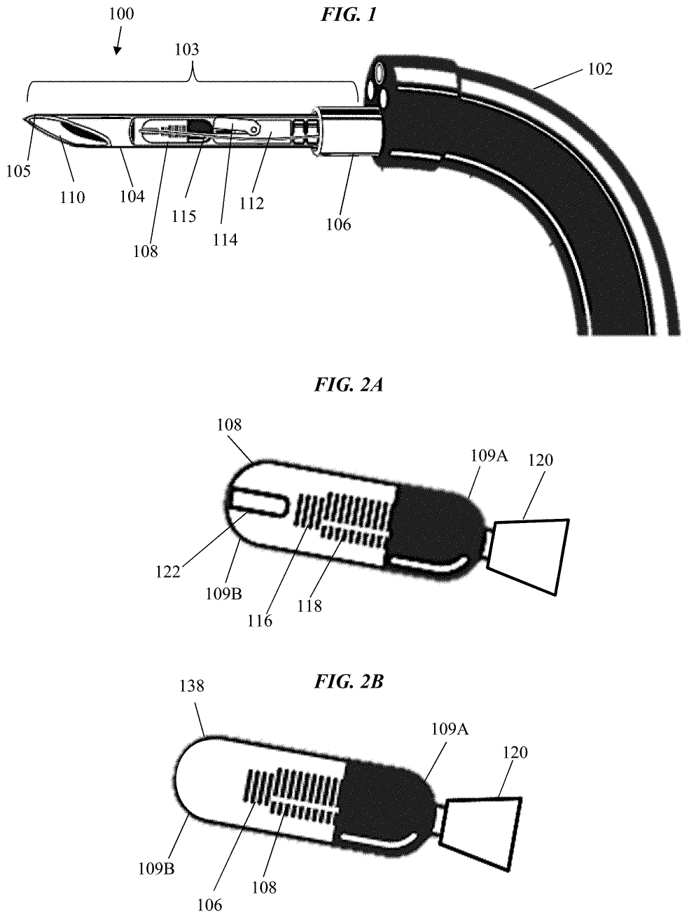

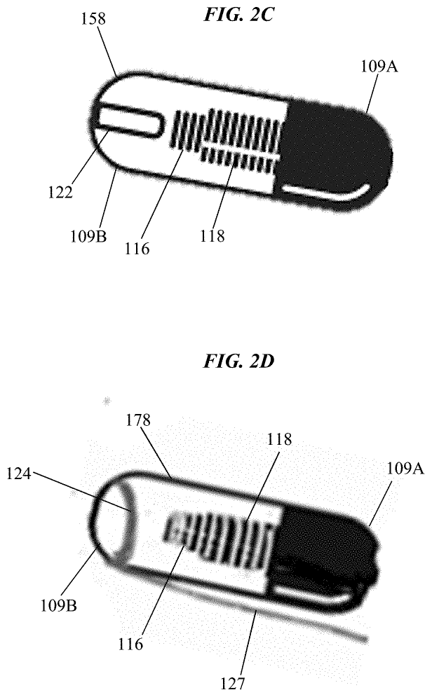

As indicated above, a marker capsule can be slidably disposed within the tube 103, e.g., within the needle 104. The marker capsule can have a variety of configurations. FIGS. 2A-2D illustrate various embodiments of marker capsules 108, 138, 158, and 178. In general, each marker capsule is pill-shaped, giving the marker capsule a longer length compared to its width. Additionally, each marker capsule can include a transmission antenna 116 and a memory 118. The memory 118 can be configured to store readable information about the location of the marker capsule, such as the reason why the marker was implanted, or the date of implantation. The transmission antenna 116 can be communicatively connected to the memory 118 in order to transmit the data wirelessly from the marker capsule 108 after insertion, and can be an RFID antenna, or a short-link antenna, such as Bluetooth®. Due to the nature of electronic devices being arranged within the marker capsule 108, the capsule itself is sealed to prevent moisture or bodily contaminants from entering the marker capsule 108 while inserted.

In addition to having internal electronics within the marker capsule, FIGS. 2A-2D also illustrate various arrangement features on the exterior surface of each marker capsule to aid in rotation of the marker capsule. The arrangement features can be positioned at various locations on the marker capsule, such as on distal or proximal ends, and each of the arranging features can be used individually or in combination in order to facilitate rotation of the marker capsule.

In the embodiment illustrated in FIG. 2A, the marker capsule 108 includes a fin 120 and a notch 122. By including more than one arranging feature, the marker capsule can be controlled from either end during the insertion procedure. In this embodiment, the fin 120 is in the form of a protrusion extending outward from a proximal end 109A of the marker capsule 138. The illustrated fin 120 is generally flat, and can thus be grasped by a grasper 114 on a driver member 112, as will be discussed in more detail below.

In addition to the fin 120, a notch 122 can be arranged on the distal end 109B of the marker capsule 108 to also aid in stabilization and rotation of the marker capsule 108. As shown, the notch 122 is in the form of a channel formed in an exterior surface of the distal end of the marker capsule. The notch has a configuration that allows a thread to be arranged within the notch 122, as shown in FIG. 1. The thread 115 can include two leading ends which pass back through the lumen 110 of the cannulated tube 103, or the thread 115 can be arranged in a working channel of the insertion device 102. These two leading ends can be used for control and rotation of the marker capsule 138 depending on the amount of tension on each leading end.

As indicated above, the marker capsule can include any number of arranging features. By way of non-limiting example, FIG. 2B illustrates a marker capsule 138 including only the fin 120, and FIG. 2C illustrates an embodiment of the marker capsule 158 including only the notch 122.

In another embodiment, the arranging feature can be in the form of a threaded loop 124 arranged on the marker capsule 108 for stabilization and to facilitate rotation of the marker capsule 108. FIG. 2D illustrates a marker capsule 178 having a threaded loop 124 arranged on a distal end 109B of the marker capsule 178. As shown, the threaded loop 124 is wrapped around an end of the marker capsule and has a leading end 127 extending therefrom. While not shown, the threaded loop 124 can include two leading ends on either side of the threaded loop 124. The threaded loop 124 is formed such that the loop diameter is smaller than the diameter of the marker capsule 178, such that when the threaded loop 124 is arranged on the distal end 109B of the marker capsule 178, the threaded loop 124 will be able to provide tension on the marker capsule without sliding down the length of the marker capsule 178.

Referring back to FIG. 1, the marker system can also include a driver member configured to advance the capsule out of the distal end of the tube 103, and optionally configured to facilitate rotation of the marker capsule. As shown the driver member 112 can include a grasper 114 at the distal end thereof having a pair of opposed jaws. The grasper 114 can be configured to grasp onto the marker capsule 108 during an insertion procedure. A person skilled in the art will appreciate that the various features on each of the disclosed embodiments can be used in any combination and in connection with any of the disclosed marker systems.

Various methods for inserting the marker capsule are also provided herein. In general, a bleb is created in a tissue surface in order to create a space to insert the marker capsule. The marker capsule is then inserted within the bleb and rotated to prevent backing out of the marker capsule. The bleb can then be sealed shut to further prevent backing out of the marker capsule.

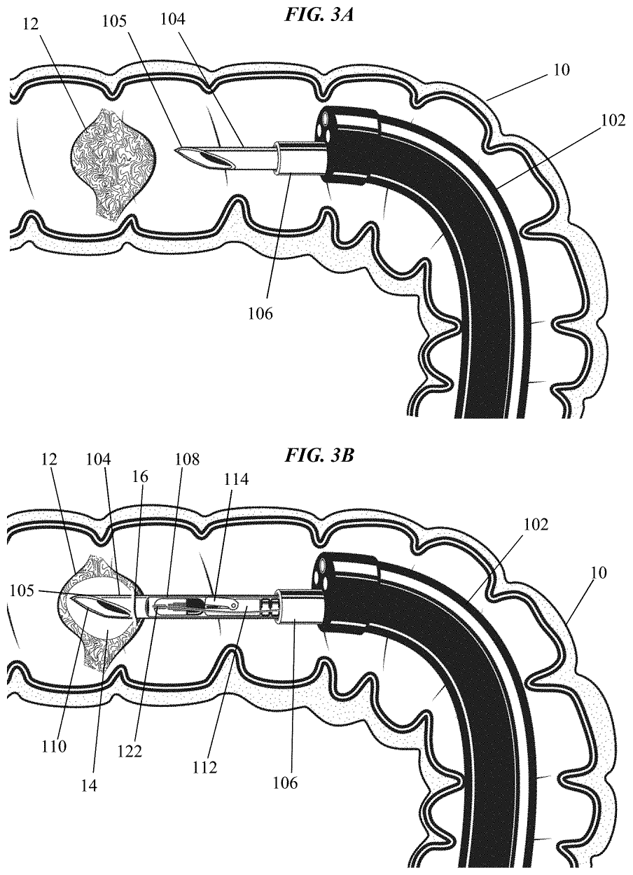

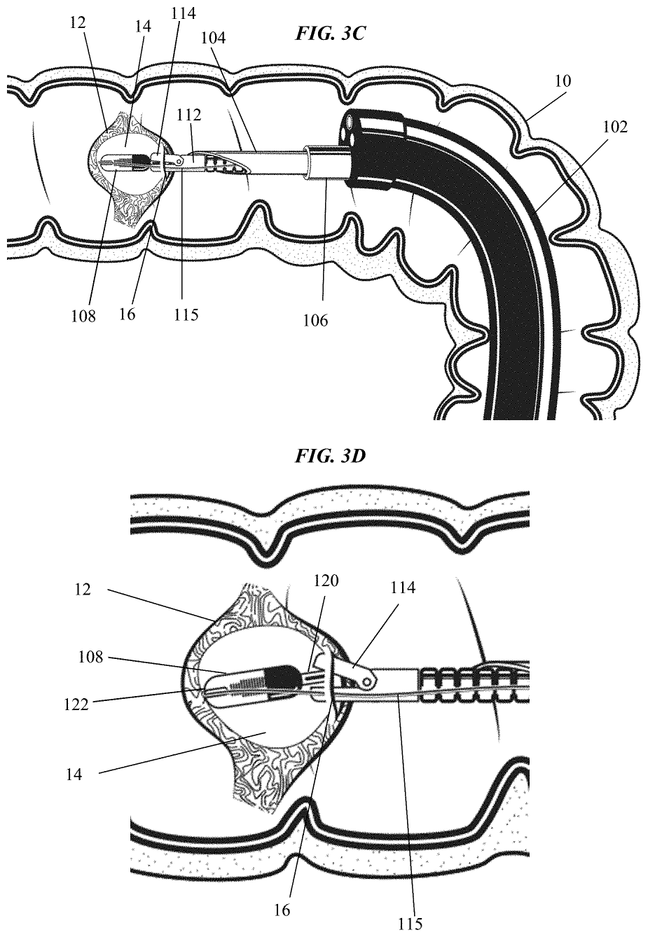

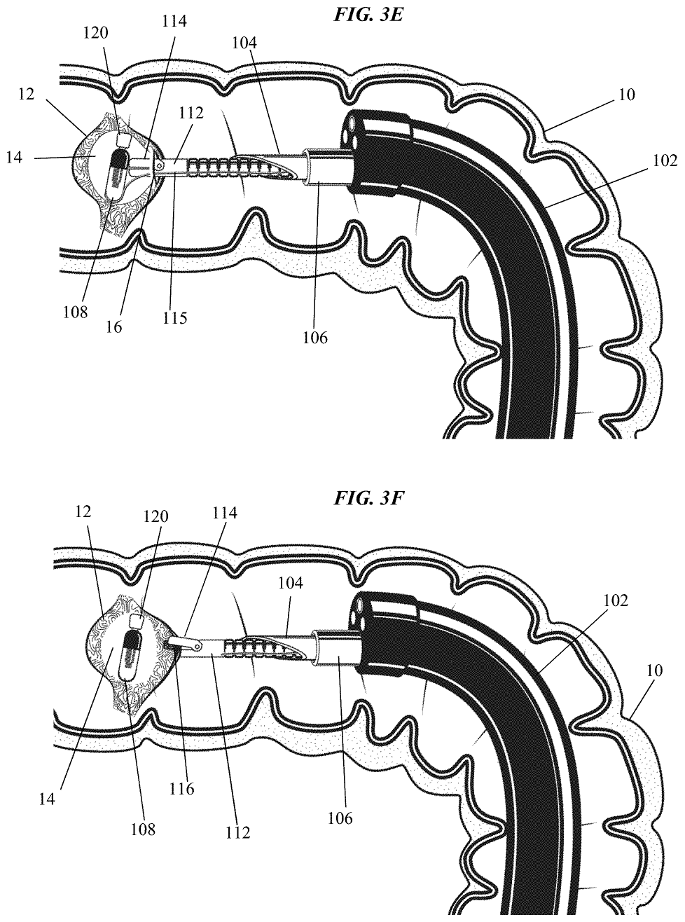

FIGS. 3A-3F illustrate a method of inserting a marker capsule 108 within tissue 10. As shown in FIG. 3A, the insertion device 102 can be inserted through a bodily cavity, such as a colon, in order to reach the desired insertion site 12. Once at the insertion site 12, the tube 103 can be advanced out of the distal end of the insertion device 102, and the cannulated needle 104 can be extended from the outer shaft 106. Using the distally arranged piercing feature 105, the cannulated needle 104 can pierce the tissue surface at the insertion site 12 to create a working portal 16. As illustrated in FIG. 3B, a fluid can be delivered through the needle 104 to the insertion site 12 to create a bleb 14. In this embodiment, a fluid, such as a saline solution, can be flowed through the lumen 110 of the cannulated needle 104 into the bleb 14 to increase the size of the bleb 14.

As depicted in FIG. 3C, after creation of the bleb 14, the needle 104 can be retracted into the outer shaft 106, and the marker capsule 108 can be inserted into the bleb 14 through the working portal 16. The driver member 112, which is arranged proximally to the marker capsule 108 within the lumen 110, can be actuated to distally displace the marker capsule 108 out of the lumen 110 and into the bleb 14 through the working portal 16. As the marker capsule 108 is being distally displaced, the grasper 114 of the driver member 112 can grasp the fin 120 of the marker capsule 108 to keep the capsule stable and aligned during the insertion. Additionally or alternatively, the thread 115 can be secured within the notch 122 of the marker capsule 108 and tensioned on both free ends to keep the marker capsule 108 stable during the insertion.

Once the marker capsule 108 is ejected from the distal end of the tube 103, the marker capsule 108 can be rotated within the bleb 14. As illustrated in FIGS. 3D-3E, the grasper 114 can release the fin 120 of the capsule 108 in order to allow rotation of the capsule 108. With the fin 120 released from the grasper 114, the thread 115 can be tensioned in order to rotate the marker capsule 108. As the thread 115 is tensioned, the thread 115 will slide within the notch 122 and begin to rotate the marker capsule 108 within the bleb 14 due to the friction between the thread 115 and the notch 122. Additionally or alternatively, the grasper 114 can be used to gradually push the marker capsule 108 into a rotated position by pushing on the fin 120. The marker capsule 108 can be rotated such that the length of the marker capsule 108 is perpendicular to the insertion direction. Since the length of the marker capsule 108 is greater than the length of the working portal 16, the marker capsule is prevented from backing out of the portal. In order to back out, the marker capsule 108 would need to rotate to align the width of the capsule with the insertion direction in order to allow the marker capsule 108 to fit through the working portal 16.

After the marker capsule 108 is fully rotated within the bleb 14, the driver member 112 is removed from the bleb. As illustrated in FIG. 3F, the bleb 14 can be sealed closed. In this embodiment, the grasper 114 can include cauterization probes arranged on the grasper 114 in order to seal the working portal 16 of the bleb 14. By sealing the bleb 14 closed, this further prevents backing out of the marker capsule 108.

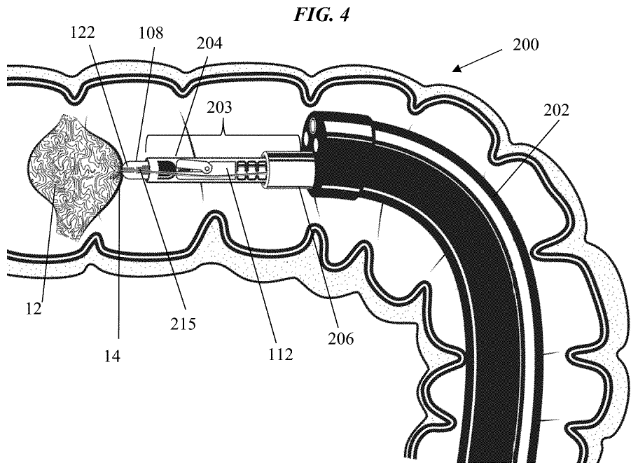

In other embodiments, the marker system can be configured to penetrate into a bleb without the use of a needle and without delivering fluid into the bleb. FIG. 4 illustrates a marker system 200 that is similar to the marker system 100 described above, and that includes an insertion device 202 having a cannulated tube 203. The cannulated tube 203 can includes an outer shaft 206 and a needle 204 extending therethrough. A marker capsule 108 is removably arranged within the cannulated tube 203. Additionally, a driver member 112 is arranged within the cannulated tube 203, proximal to the marker capsule 108. However, in this embodiment the needle 204 lacks a piecing feature and instead is merely an elongate tube. In order to create the working portal in the tissue 10, a thread 215 can be arranged within a notch 122 in the marker capsule and it can be coupled to a power supply and configured to apply an electrical current to the tissue. In this embodiment, the thread 215 is abutted against the tissue and is electrified. The electrical current passing through the thread 215 can ablate the tissue to create a working portal through the tissue and into the bleb. The method of insertion and rotation of the marker capsule 108 is in the same manner as previously described in relation to FIGS. 3C-3F.

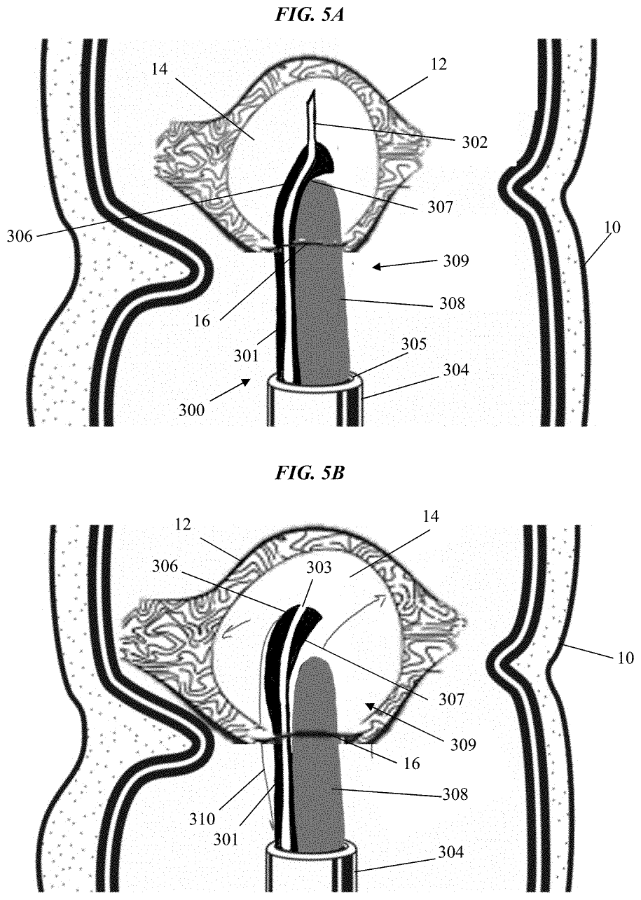

In other embodiments, the rotation of the marker capsule can be caused automatically when the marker capsule is pushed distally from the cannulated tube. FIGS. 5A-5B illustrate a marker system 300 which can automatically rotate the marker capsule when the capsule is ejected from the cannulated tube. As shown, the marker system 300 includes a cannulated tube having an outer shaft 304 and an inner shaft 301 extending through the outer shaft 304. The inner shaft 301 can be a flexible member which can be arranged within the lumen 305 of the outer shaft. The inner shaft 301 can include a hook-shaped member 306 hat the distal end thereof. The hook-shaped member 306 can be formed by forming the inner shaft 301 with a rounded or bullet-shaped closed distal end, and by providing a cut-out or opening 309 in a sidewall of the inner shaft 301 adjacent to the distal end for allowing the capsule to exit from the inner shaft 301. The internal curved surface 307 can curve towards the cut-out 309 in the outer shaft 304.

Additionally, the inner shaft 301 can include a lumen 303 formed through a sidewall thereof and having a needle 302 slidably disposed therein. The needle 302 can be flexible and can thus bend within the lumen 303. The needle 302 can be extended from and retracted into the lumen 303, and can be used to pierce and create the bleb 14 within the tissue 10 in the same manner as described above. Additionally, the needle 302 can be removed from the lumen 303 during the insertion of the marker capsule 308 in order to make the inner shaft 301 and the hook-shaped member 306 more flexible. A marker capsule 308 can be arranged within the lumen 305 of the outer shaft 304. A drive member, similar to those described above, can be arranged proximally within the lumen 305 and configured to distally displace the marker capsule 308 out of the lumen 305.

As illustrated in FIG. 5B, as the driver member is advanced distally in order to push the marker capsule 308 out of the lumen 305, the curved surface 307 will cause the marker capsule 308 to rotate while being inserted into the bleb 14. The rotation of the marker capsule 308 can occur after the marker capsule 308 has passed through the working portal 16. In addition to pushing the marker capsule 308 out of the lumen 305, a thread 310 can be attached to the outer surface of the hook-shaped member 306 in order to aid in the release of the marker capsule 308 from the outer shaft 304.

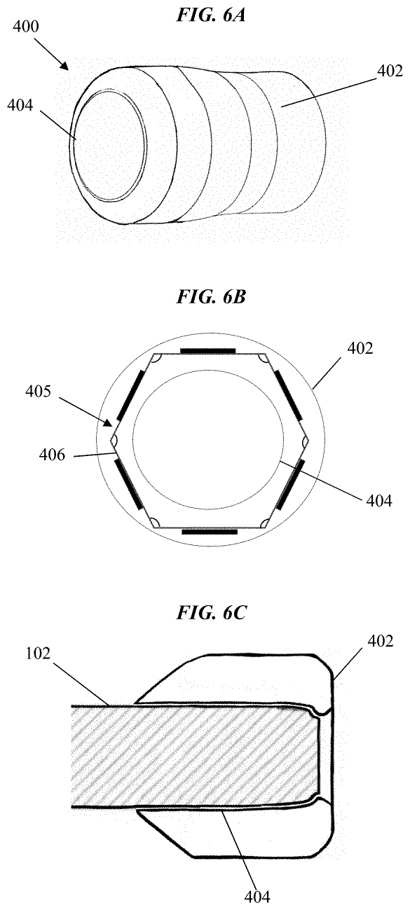

After insertion of the marker capsule is complete, it may be advantageous to relocate the marker capsule during a subsequent procedure. In order to locate the marker capsule, a receiver member arranged on an insertion device may be used. As illustrated in FIGS. 6A-6C, a cap assembly 400 can be used to receive data from the transmission antenna 116 of the marker capsule 108. The cap assembly 400 can include a water proof housing 402 including a thru-bore 404 arranged within the housing 402. In order to communicate with the marker capsule 108, a receiver member 405 can be arranged within the cavity of the cap assembly 400. In this embodiment, the receiver member 405 can include a plurality of flexible circuit boards 406 formed into a compatible shape in order to fit within the cavity of the cap assembly 400. Additionally, the receiver member can have a short-range transmission receiver and a long-range transmission antenna. The short-range transmission receiver can be an RFID receiver to connect with the transmission antenna 116 within the marker capsule 108. The short-range transmission receiver can be used to locate the marker capsule 108 within the insertion site without the need for visual markers. Once located, the data set contained within the memory 118 can be transmitted to the short-range transmission receiver of the CPU. The data can then be transmitted outside of the patient's body using the long-range transmission antenna, such as a Bluetooth® connection to a computer outside of the body. In this embodiment, the cap assembly 402 can be arranged on an insertion device 102 using the thru-bore 404. The insertion device can be press fit onto the end of the insertion device 102, and can be held in place via a friction fit between the housing 402 and the insertion device 102.

Certain exemplary implementations have been described to provide an overall understanding of the principles of the structure, function, manufacture, and use of the systems, devices, and methods disclosed herein. One or more examples of these implementations have been illustrated in the accompanying drawings. Those skilled in the art will understand that the systems, devices, and methods specifically described herein and illustrated in the accompanying drawings are non-limiting exemplary implementations and that the scope of the present invention is defined solely by the claims. The features illustrated or described in connection with one exemplary implementation may be combined with the features of other implementations. Such modifications and variations are intended to be included within the scope of the present invention. Further, in the present disclosure, like-named components of the implementations generally have similar features, and thus within a particular implementation each feature of each like-named component is not necessarily fully elaborated upon.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

One skilled in the art will appreciate further features and advantages of the invention based on the above-described implementations. Accordingly, the present application is not to be limited by what has been particularly shown and described, except as indicated by the appended claims. All publications and references cited herein are expressly incorporated by reference in their entirety.

What is claimed is:

1. A marker system, comprising:
an elongate cannulated tube having a lumen extending therethrough and a piercing feature arranged at a distal end thereof that is configured to create a working portal through tissue such that fluid can be delivered into the tissue to form a bleb;
a marker capsule removably disposed within the lumen of the cannulated tube, the marker capsule including a transmission antenna, and the marker capsule having an arranging feature disposed on an external surface thereof configured to facilitate rotation of the marker capsule within the bleb; and
a driver member arranged within the lumen of the cannulated tube proximal to the marker capsule, the drive member being configured to advanced distally to displace the marker capsule through the working portal and into the bleb.

2. The marker system of claim 1, wherein the arranging feature comprises a fin extending from the external surface of the marker capsule.

3. The marker system of claim 2, wherein the driver member includes jaws configured to grasp the fin to rotate the marker capsule.

4. The marker system of claim 1, wherein the arranging feature comprises a notch arranged on a distal end of the marker capsule and a thread arranged within the notch and configured to apply a tension to the marker capsule to rotate the marker capsule.

5. The marker system of claim 1, wherein the arranging feature comprises a threaded loop arranged on a distal end of the marker capsule and configured to apply tension to the marker capsule to rotate the marker capsule.

6. A marker system, comprising:
an elongate cannulated tube having a lumen extending therethrough, a distal end of the tube including a hook-shaped member with an internal curved surface;
a marker capsule removably disposed within the lumen of the cannulated tube, the marker capsule including a transmission antenna therein;
a driver member arranged within the lumen of the cannulated tube proximal to the marker capsule, the driver member being configured to advanced distally to advance the marker capsule along the internal curved surface of the hook-shaped member such that the marker capsule is caused to rotate; and
a cannulated needle arranged along the elongate cannulated tube and configured to be slidably advanced from a distal end of the elongate cannulate tube to penetrate tissue.

7. The marker system of claim 6, wherein the cannulated needle is slidably disposed through a second lumen formed in a sidewall of the cannulated tube.

8. The marker system of claim 6, wherein the hook-shaped member is positioned to block distal advancement of the marker capsule, and wherein the hook-shaped member is flexible to deflect when the marker capsule is advanced distally.

9. The marker system of claim 8, further comprising a tether coupled to the hook-shaped member and configured to apply a force to the hook-shaped member to cause the hook-shaped member to deflect and thereby allow distal advancement of the marker capsule.

10. A marker system, comprising:
an elongate cannulated tube having a lumen extending therethrough;
a marker capsule removably disposed within the lumen of the cannulated tube, the marker capsule having a notch formed in a distal surface thereof and a transmission antenna therein;
a thread arranged within the notch; and
a driver member arranged within the lumen of the cannulated tube proximal to the marker capsule, the drive member being configured to distally advance to displace the marker capsule from the distal end of the cannulated tube.

11. The marker system of claim 10, wherein the thread is coupled to an energy source and is configured to apply an electrical energy to a tissue surface to create a working portal.

12. The marker system of claim 10, wherein the thread is configured to apply tension to the marker capsule to rotate the marker capsule.

13. The marker system of claim 10, further comprising a receiver member have a short-range transmission receiver and a long-range transmission antenna, the receiver member configured to receive a data set from the marker capsule via the short-range transmission receiver, and to transmit the data set via the long-range transmission antenna.

14. The marker system of claim 13, wherein the receiver member is disposed within a water-proof cap configured to couple to an end of an endoscope.

15. The marker system of claim 14, wherein the short-range transmission receiver is a RFID receiver, and the long-range transmission is a short-link radio antenna.

16. A method for implanting a marker in tissue, comprising:
inserting an elongate cannulated tube into a tissue surface to create a working portal through the tissue surface;
injecting a fluid through the cannulated tube into the tissue surface to create a bleb;
ejecting a marker capsule from a distal end of the cannulated needle into the bleb, the marker capsule having a transmission antenna therein; and
rotating the marker capsule within the bleb such that a length of the marker capsule extends adjacent to the working portal to prevent passage of the marker capsule through the working portal.

17. The method of claim 16, wherein the fluid is injecting through a cannulate needle extending through the cannulated tube.

18. The method of claim 16, further comprising, prior to inserting the elongate cannulated tube into a tissue surface, advancing a needle from a distal end of the cannulated tube.

19. The method of claim 16, wherein the cannulated needle is removed from the bleb prior to rotation of the marker capsule.

20. The method of claim 16, wherein rotating the marker capsule comprises applying tension to a tether coupled to the marker capsule to cause the marker capsule to rotate.

21. The method of claim 16, wherein rotating the marker capsule comprises grasping a fin on the marker capsule with a grasper and manipulating the grasper to rotate the marker capsule.

22. The method of claim 16, further comprising positioning a receiver member adjacent to the marker capsule such that the receiver member receives a data set from the marker capsule via the transmission receiver.
